(19) United States
(12) Patent Application Publication
(10) Pub. No.: US 2009/0070139 A1
(43) Pub. Date: Mar. 12, 2009

(54) METHODS AND DEVICES FOR MAINTAINING A DIET FREE FROM REFINED FOODS

(76) Inventor: Joan Rewbridge Ifland, Houston, TX (US)

Correspondence Address: FOLEY & LARDNER LLP, 111 HUNTINGTON AVENUE, 26TH FLOOR, BOSTON, MA, 02199-7610, US

(21) Appl. No.: 12/168,713

(22) Filed: Jul. 7, 2008

Related U.S. Application Data
(60) Provisional application No. 60/990,414, filed on Nov. 27, 2007.
(60) Provisional application No. 60/949,491, filed on Jul. 12, 2007.

(51) Int. Cl. G06Q 50/00; G06Q 30/00; A61B 5/00

(52) U.S. Cl. 705/2; 705/26; 600/300

(57) ABSTRACT

Described herein are methods, apparatuses and kits suitable for treating a person afflicted with a disorder related to the consumption of refined foods.

### BACKGROUND OF THE INVENTION

Refined foods have addictive properties that create behaviors and conditions that cause or contribute to many different conditions, such as obesity, pain, diabetes, hypertension, cardiovascular disease, depression, epilepsy, lack of mental acuity and physical energy, and other health disorders. Several studies support the theory that food can be addictive. A number of studies, for example, show that when abusers of alcohol and nicotine enter recovery, they gain weight. The interpretation of this phenomenon is that food can stimulate the same reward neuropathways as alcohol and nicotine. Not surprisingly, one of the pharmaceutical treatments for alcoholism, naltrexone, also inhibits sucrose cravings.

Addictive behavior appears to be facilitated by the release of the neurotransmitter dopamine. The degree of the dopamine response from food does not appear to be as great as that of drugs. Nonetheless, the region of the brain that is affected by the release of dopamine is the same for both drugs and refined foods. There is also evidence in animal studies that visual and olfactory cues can stimulate addictive food responses in the form of dopamine releases.

The addiction to refined foods makes abstaining from refined foods very difficult, with some of the same issues that make the breaking of addiction to other substances like tobacco or heroin very difficult. These issues include the difficulty in countering the symptoms of physical craving for the addictive foods, the difficulty in breaking the habit of eating these addictive foods, especially where various environmental cues trigger recurrence of the habit, the feelings of mental lethargy that come from eating refined foods and that make it that much harder to focus on maintaining a diet, the cultural acceptance of the routine consumption of refined foods, and the reluctance to acknowledge an addiction to these foods.

Methods of managing refined food addiction have not been able to overcome the issues inherent in treating this addiction. Diets that attempt to eliminate refined foods are difficult to follow, since a person would have difficulty identifying just what particular foods to avoid. In addition, since refined foods are so ubiquitous, and unrefined replacement foods can be difficult to find, it is also difficult to avoid these foods yet still get proper nutrition. Furthermore, the addiction to these foods causes withdrawal symptoms when a person attempts to abstain from them, making a relapse more likely. A person who does not know the symptoms of addiction to refined foods, or cannot observe or measure these symptoms as he or she experiences them, is less motivated to follow a diet plan. A further complication is that when a person purchases food they are often subjected to advertising, packaging, and displays that serve as visual cues that trigger their addictive cravings for refined foods. Restrictive diets also often lack the flexibility to be adapted to a person's individual food preferences and caloric needs.

There is a need for methods and apparatuses for treating refined food addiction and the attendant conditions caused by this addiction. Such methods and apparatuses would make it easier to maintain a diet free from refined foods and to motivate compliance. Methods and apparatuses that make the procurement and preparation of non-refined foods easier, as well as methods and apparatuses that measure the symptoms caused by the presence and absence of refined foods so a person can modify their behavior accordingly, would help in achieving a diet free from refined foods. Methods to distinguish addictive foods from non-addictive foods would also be useful to determine the appropriate number of calories to consume. The methods and apparatuses would need to overcome factors that tend to hinder adherence to a non-refined food diet, such as the presence of triggers in food or the environment that encourage the consumption of refined foods, the reduction of pleasure producing neurotransmitters brought about by withdrawal from refined foods, and the reduction in blood glucose levels caused by the consumption of refined foods.

### SUMMARY OF THE INVENTION

The present invention relates to methods and devices for initiating and maintaining a diet free from refined foods that overcomes the problems inherent in treating an addiction, as well as those problems that are caused by the addiction to refined foods itself.

In one embodiment, the present invention is directed to a method for treating a person afflicted with a disorder related to the consumption of refined foods, comprising: using a food kit comprising prepared food that does not contain refined food products, wherein the kit comprises sufficient food to supply the nutritional requirements of the person during a specified period of time; and eliminating the consumption of refined food products during the period of time, thereby treating the person afflicted with the disorder related to the consumption of refined foods. In a particular embodiment, the disorder is Refined Food Addiction. In another embodiment, the refined food product is selected from the group consisting of: plant products that have been concentrated, plant products that have been made into a powder, plant products that have been made into a syrup, plant products that have been made into a crystal, and combinations thereof. In yet another embodiment, the method comprises the elimination of food products and additives selected from the group consisting of: salt, caffeine, fats exposed to high temperatures, and processed dairy products. In a further embodiment, the food kit comprises an apparatus that organizes prepared unrefined foods into storage compartments that separate food items into individual meals. In one embodiment of this method, each item of a particular type of food is enclosed in an individual container. In yet another embodiment of this method, each container is color-coded for the particular type of food, wherein the kit comprises instructions for matching the color-coded containers to create meals by combining particular types of food. In another embodiment the method further comprises a machine-readable bar code on each container. In a further embodiment the food kit further comprises optional seasonings. In one particular embodiment the food kit comprises specifically apportioned meals. In a further embodiment of the method, each meal is apportioned according to a static ratio of proteins to fruit, vegetables and starches. In still another particular embodiment of the invention, the method further comprises assessing eating behaviors during the period of time using Diagnostic and Statistical Manual (DSM), International Statistical Classification of Diseases and Related Problems (ICD), or any other substance use disorder criteria; obtaining responses to an assessment questionnaire that ranks the severity of addictive behavior using DSM, ICD, or any other substance use disorder criteria as applied to the consumption of edible substances at the beginning of the method, at a later time point, and after the consumption of any refined food, comprising the recording of the responses to the questionnaire previously obtained; and comparing the responses given to questionnaire at the beginning of the method to those given at later time periods. A further embodiment of the method further comprises a means to reorder the food kit when food is almost exhausted. In an additional embodiment, the method further comprises a means to alter the order of the initial food kit package in accordance to the food preferences of the person. Yet another embodiment further comprises one or more health monitoring devices. Still another embodiment further comprises one or more health monitoring devices. Another embodiment further comprises the use of a wireless, hand-held electronic device. In a further embodiment, the hand-held electronic device has telecommunication and computational capabilities. In yet another further embodiment, the hand-held electronic device performs one or more of the functions selected from the group consisting of: collecting data on food consumption, automatically reordering food kit packages when a previous food kit is close to exhausted, showing financial savings from the use of the food kit, providing ratings of restaurants and grocery stores, providing information on recommended meals for a particular diet, providing motivational feedback on the positive consequences of abstaining from refined foods, providing motivational feedback on the negative consequences of relapse in consuming refined foods and providing information on food allergies. One embodiment of the invention further comprises the use of adjustable clothing to measure waist and hip, wherein the clothing comprises goals displayed in the lining of the fabric in place of numbers. In another embodiment, the food kit comprises food with adjustable fat levels. In still another embodiment, the food kit comprises means suitable for a rotation or vegan diet. An additional embodiment further comprises a carbohydrate blocker. Another embodiment of the method further comprises an apparatus for exercise. Yet another embodiment further comprises a stress monitor to be used in combination with meditation. One particular embodiment of the method also further comprises dietary supplements.

One embodiment of the present invention is directed to a method for providing a food kit for treating an eating-related substance use disorder; comprising using an apparatus that, in response to a customer order, organizes prepared, unrefined foods into a food kit, wherein the food kit comprises multiple compartments that separate prepared foods into separate meals, and wherein the food kit comprises enough meals for a specified period of time; and a means for the customer to order the food kit; and delivery of the food kit to the customer whereby appropriate use of the delivered food kit treats the eating-related substance use disorder. In a further embodiment of the invention the food kit is delivered overnight. Yet another embodiment further comprises a means for the customer to reorder the food kit when food is almost exhausted. A further embodiment of this method comprises providing health monitoring equipment. In still another embodiment, the health monitoring equipment comprises a blood pressure cuff. In a further embodiment, the health monitoring equipment comprises a heart rate monitor. In yet another embodiment, the health monitoring equipment comprises blood glucose measuring equipment. In a still further embodiment, the health monitoring equipment comprises a cholesterol measuring device. In an additional embodiment, the health monitoring equipment comprises a device to measure weight and percentage of body weight represented by fat. A further embodiment of the method further comprises instructions for eating foods from the kit according to a daily calendar. In an additional embodiment of the method, the means for the customer to order the food kit is provided on an internet website.

The present invention is also directed, in one embodiment, to a method for identifying a disorder related to the consumption of refined foods comprising one or more methods selected from the group consisting of: obtaining responses to an assessment questionnaire, evaluating a medical history, detecting disorder-related biomarkers, and neuroimaging, wherein the size of the dopamine field is examined to detect abnormalities.

Another embodiment of the present invention is directed to a method for measuring the addictive characteristics of a food, comprising measuring of degree of food addiction using an assessment questionnaire; adhering to a diet that eliminates all refined food items for a specified period of time; measuring symptoms of food addiction after the specified period of time on the restricted diet; reintroducing a single refined food item after the specified period of time; measuring symptoms of food addiction at a time point after a single refined food is reintroduced; re-eliminating all refined foods from the diet; re-measuring symptoms of food addiction at a time point after all refined foods have again been eliminated from the diet; and comparing the measurements of symptoms of food addiction, thereby determining the severity of the addictive property of the specific refined food tested.

Another embodiment of the present invention is directed to an apparatus that organizes prepared unrefined foods into a food kit wherein storage compartments separate food items into individual meals sufficient to last during the period of time. In another embodiment of this apparatus, each item of a particular type of food is enclosed in an individual container. In still another embodiment of this apparatus, each container is color-coded for the particular type of food with instructions for matching the color coded containers to create meals by combining particular types of food. A additional embodiment of this apparatus further comprises a machine-readable bar code on each container. In still another embodiment, the food kit further comprises optional seasonings. In yet another embodiment, the food kit comprises specifically apportioned meals. In a further embodiment, each meal is apportioned according to a static ratio of proteins to fruits, vegetables and starches.

One embodiment of the present invention is directed to adjustable clothing comprising goals displayed in the lining of the fabric in place of numbers to measure the waist and hip, thereby providing motivation for weight loss and a means for monitoring weight loss.

Another embodiment of the present invention is directed to a method for treating adult epilepsy, comprising using a food kit comprising prepared food that does not contain refined food products such that the kit comprises sufficient food to supply the nutritional requirements of the person during a specified period of time; and eliminating the consumption of refined foods during the period of time, thereby treating adult epilepsy.

An additional embodiment of the present invention is directed to a method for treating pain, comprising using a food kit comprising prepared food that does not contain refined food products such that the kit comprises sufficient food to supply the nutritional requirements of the person during a specified period of time; and eliminating the consumption of refined foods during the period of time, thereby treating pain.

Still another embodiment of the present invention is directed to a method for treating obesity, comprising using a food kit comprising prepared food that does not contain refined food products such that the kit comprises sufficient food to supply the nutritional requirements of the person during a specified period of time; and eliminating the consumption of refined foods during the period of time, eliminating the consumption of refined foods during the period of time, thereby treating obesity.

Yet another embodiment of the present invention is directed to a method for treating diabetes, comprising using a food kit comprising prepared food that does not contain refined food products such that the kit comprises sufficient food to supply the nutritional requirements of the person during a specified period of time; and eliminating the consumption of refined foods during the period of time, thereby treating diabetes.

### DETAILED DESCRIPTION OF THE PREFERRED EMBODIMENTS

The present invention relates to methods and devices for initiating and maintaining a diet free from refined foods that overcomes the problems inherent in treating an addiction, as well as those problems that are caused by the addiction to refined foods itself. The invention achieves this result by stabilizing blood glucose, stabilizing pleasurable neurotransmitters, reducing exposure to triggers, and eliminating triggering substances. Described herein are, for example, methods for treating a person afflicted with disorders related to the consumption of refined foods, methods for providing a food kit for treating such disorders, methods for identifying a disorder related to the consumption of refined foods, methods for measuring the addictive characteristics of various foods, apparatuses that organizes prepared unrefined foods into a food kit, adjustable clothing to measure and motivate progress on the diet, methods for treating adult epilepsy, methods for treating pain, methods for treating obesity, and methods for treating diabetes.

When a person is dealing with an addiction, it is important that the person abstain from the addictive substance, or else they will continually trigger the addiction whenever they consume the addictive substance. In disorders related to the consumption of refined foods, individuals find it very difficult to completely abstain from refined foods, due to the difficulty in knowing whether a food contains refined food ingredients, and the constant presence of refined foods in grocery stores and restaurants, which become a temptation while food shopping or going out to eat. Grocery stores and restaurants can trigger customers into overeating addictive foods by the prominent availability of these foods, and by visual and olfactory cuing of the addictive behavior.

The degree and extent of food addiction can be measured with techniques used for other more commonly known addictions such as those with drugs and alcohol. For example, the DSM dependence criteria, created by the American Psychiatric Association for the measurement of addiction disorders, has been applied with validity and reliability to eating behavior. When this dependence criteria is applied to the consumption of refined food, addictive dependence is demonstrated for refined food use. The ICD, an international version of the DSM, is one example of another substance use disorder criteria that can be applied to eating behavior. Other substance use disorder criteria can be used as well.

The food kit of the invention helps overcome these obstacles by providing all the food necessary for an individual for a specified period of time, herein defined as a period of time sufficient to begin to feel the effects of recovery from the addiction. For example, a sufficient period of time can be about 3 days to about 1 month, or about 5 days to about 2 months, or about 1 week to about 2 weeks, or about 3 weeks to about 1 month, though about a one week supply of food is a likely quantity for the food kit due to storage and shipping considerations. Since the individual has all their dietary needs addressed by the food kit they do not need to go shopping for food and thus they avoid temptation from the presence of refined food at the store, as well as the packaging, advertising, and promotional displays for such food found in food stores. The food kit meets the nutritional requirements of the individual for this specified period of time. The phrase “nutritional requirements” is used herein to mean the calories and nutrients necessary to ensure good health as recommended by recognized authorities in the field of human nutrition.

It is an object of the methods herein, comprising the use of a food kit and the elimination of refined foods from the diet for the specified period of time, to treat a refined food addiction. “Refined food addiction” is used herein to mean a disorder where a person exhibits substance dependence behaviors with regard to refined foods in accordance to criteria found, for example, in the DSM, or, for example, in the ICD. “Refined foods,” as used herein, refer to products that have been concentrated, or made into a powder, syrup or crystal. Examples of refined foods include, but are not limited to, products that contain flour or table sugar, such as, for example, bread or ice cream. Refined foods can be plant products, for example. Refined foods also include food products that contain ingredients such as, for example, salt, caffeine, fats exposed to high temperatures or processed dairy products. Examples of these types of food include, but are not limited to, fried foods, regular coffee and cheese.

The food kit has several features that help improve adherence to the non-refined food diet by making the organization and preparation of these specialized meals easier. The food kit contains containers of prepared non-refined food, such containers can be, for example, plastic packets, wax paper packets, cardboard packets, foil packets or similar packages that can be useful in storage or to protect the freshness of the food. The containers of non-refined food require minimal preparation, usually just heating, so that the person does not have to prepare their own food with the risk of using refined food ingredients. In addition, the food kit has storage compartments that separate the food containers into individual meals, so the person can assemble meals easily using only the prepackaged unrefined food products. Each container of the food kit contains food of a particular category such as, for example, protein, vegetables, starch and fruit. The containers can be color coded, with a given color representing the components of a meal such as breakfast or lunch, which allows the person to create the meals without resorting to refined food products out of habit or convenience. In addition, the color coding and the packaging of the food into discrete containers allows the person flexibility in creating meals in order to create the variety sometimes needed to defeat boredom from a restricted diet.

Each container of food can have a machine readable bar code. This bar code allows a person to record food consumption by scanning the bar code on the container with an appropriate scanning device. This information can be useful for a variety of purposes, such as, for example, automatically reordering food kits when scanning indicates most food in previous kit has been consumed, measuring progress in adhering to the diet, or other purposes related to measurements of the consumption of the non-refined food included in the food kit.

The food kit can also contain optional seasonings that can be used to season the meals within the food kit. Such seasonings also help defeat the boredom that sometimes occurs from a restricted diet. In addition these seasonings allow the food to be made more appetizing according to the individual preference of the person using the food kit.

The composition of the individual food packets of the food kit also help adherence to the non-refined food diet. Each packet of the food kit contains food of a particular category such as, for example, protein, vegetables, starch and fruit which are packed in specifically apportioned meals, herein defined as meals of small quantities of food, about 3-6 ounces. The small portion size allows for the consumption of low calorie meals, with the flexibility to increase the number of meals when the person is feeling elevated hunger due to such things as unusually high exercise activity or certain phases of the menstrual cycle. The individual is instructed to wait a set period of time, for example, about 15-20 minutes, before deciding whether to eat an additional meal, since studies have indicated that the mental experience of food satiation often occurs up to 20 minutes after eating for people with overeating disorders. In this way, the person can consume fewer calories on average and yet feel greater satisfaction, and have less of a tendency to relapse into overeating behavior due to momentary cravings.

The meals within the food kit can be apportioned, for example, according to a static ratio of proteins to fruit, vegetables and starches, herein defined as a ratio of protein to the other foods that is constant when matched to other food types, such as vegetables, starches, and fruit, in accordance with the instructions of the kit. The static ratio herein defined would also be a high enough ratio of proteins to other foods as to achieve the optimal balance of neurotransmitters for the proper mental and emotional function for the recovering refined food addict. Such a balance of neurotransmitters is important, since the lack of the stimulus from the refined food may initially lower the release of pleasurable neurotransmitters and increase the likelihood of a relapse of the addiction.

The invention uses the symptoms of addiction to refined foods as well as the beneficial effects of abstaining from refined foods as motivation to encourage adherence to the diet. Refined foods instigate the same behaviors and feelings as addiction to other substances, and these behaviors and feelings can be measured with tools used by psychiatrists and other addiction health care professionals, such as the DSM substance related disorder questionnaire, or the ICD, or any other substance use disorder questionnaire. The DSM, or ICD, or other substance related disorder questionnaires can be modified to ask questions specific to a refined food addiction, such as, for example, those questions related to whether the person needs to eat more to feel satisfied, whether the person eats to correct a low feeling, whether the person eats more than intended, whether the person has tried to cut back on their food consumption, whether the person spends a great deal of time eating and recovering from eating, whether the person has missed important activities or events such as family activities and hobbies in order to eat, and whether the person eats in spite of their knowledge of the consequences. When this questionnaire is modified to collect and measure addictive behaviors concerning refined foods, the results of the questionnaire can be used to show the person their level of addiction and how refraining from refined foods reduces and eliminates this behavior over time. When the person views the results of the questionnaire at specific periods of time during their use of the method, they are further motivated to continue to abstain from refined foods. The results of the person's answers to the questionnaire show them that they are indeed addicted to these foods. The change in the answers to the questionnaire that they record as they continue to adhere to the diet show them that they are making progress and further motivates them to continue to abstain from refined foods. The individual using this method can also compare and contrast the way they feel when using particular refined foods versus the way they feel when avoiding refined foods, and in this way they gain an appreciation of the beneficial effects on their emotional, physical, and mental functioning. The invention, in addition to being used by the person treated, can also be used by health care professionals to identify, assess, and devise treatment plans for refined food addiction.

The food kit can be reordered and shipped directly to the person when the food from the previous food kit is close to exhausted, thereby avoiding the need to go to a restaurant or food store and thus avoiding the attending temptation. Further flexibility is provided by allowing the person to specify their food preferences when ordering or reordering the food kit, thereby tailoring the food kit to the individual and ensuring the person is more satisfied with their food choices on the diet.

The invention can use health monitoring devices to indicate progressive improvement in health status, thereby further motivating compliance with the non-refined food diet and additional lifestyle changes. Often individuals will become discouraged and unmotivated when undertaking a diet program because they fail to see any noticeable progress in achieving goals such as weight reduction or improved health. The invention can use these health monitoring devices to indicate progress in health-related goals, and thus increase adherence to the diet. Such health monitoring devices can include, but are not limited to, a blood pressure cuff that also measures heart rate, blood glucose measuring equipment consisting of lancets and glucose meter, a cholesterol meter, a weight/fat scale, along with instructions for using the equipment. As improvements in the measures of health such as, for example, blood pressure and weight are experienced through using the equipment, the person becomes more motivated to continue the diet program.

The invention can contain a hand-held, wireless, electronic device with the ability to scan bar codes and store data. This device can have computational and telecommunications capabilities, similar to a Blackberry® or Treo® phone. Health and diet information would be available through downloads or by accessing a website. Partnering with a mobile phone company to adapt an existing phone to accommodate an interface with a scanning device and health care data monitors is one route to create these devices. The device will support the person on the diet in obtaining safe, non-refined food. The person can set-up food preferences, rate the meals provided with the food kit, and record data on any food allergies that the person may have. The devices can also collect data on food consumption through a scanning device that reads bar codes on food products and automatically sends a new supply of food as needed, or automatically sends voice or text message to request authorization for the next shipment of food. The device can also automatically show financial savings from purchasing the food kit versus restaurant or grocery story purchases cumulative for the last week, month, year, from the inception of the food kit program. In addition, the device can retrieve restaurant and grocery store customer-generated ratings, similar to Zagat's®, based on such things as: the intensity of triggers to use unhealthy food, refined foods highlighted at cashier, flashing promotional videos, prominent display of refined foods in magazines at cashier, any pushing of ‘free’ refined foods such as, for example, chips, hot bread, or crackers, the presence of back-lit soda machines, heavy promotion of discounted refined foods, excessive portion sizes, intense smells of hot grease or baked goods, and availability of good, unrefined food.

The device can provide information on meal preparation consistent with a non-refined food diet, such as, for example, specific recommendations for unrefined dishes, appropriate serving sizes, and appropriate pricing. In addition, the device can contain links to counseling/advice services, both text message and voice, and information on mediation and exercise. The device can motivate the person through the storage, analysis, and retrieval of personal data relating to food consumption. The device can have the capacity, through a scanning device for the food packets provided in the food kit, as well as commercial food bar codes, to record personal food consumption. Reinforcing messages on the positive consequences of abstinence from refined foods, and the negative consequences of relapse, can be provided on the device. As an added feature the device can interface directly with, for example, blood pressure, glucose, and cholesterol monitoring devices to collect health data that can be reviewed by the individual. In this way the device provides motivation to adhere to the diet as well as useful information to avoid potential obstacles to maintaining the diet.

The invention can include adjustable clothing with a hip/waist measuring system and the use of such clothing. The adjustable clothing displays messages of encouragement that create a feeling of accomplishment as the person reaches their weight and measurement goals. These messages are displayed in the lining of the fabric in place of numbers to measure the waist and hip. In this way the person is motivated by seeing their weight loss along with motivational messages associated with their health goals.

The goals displayed in the lining of the fabric can be, for example, the achievement of a reduction in the diameter of the person's waist, with such goal indicated by a positive message such as, for example, “great work, you're halfway to your target clothes size”, or, for example, “great going, scientists think waist to hip ratio is a good indicator of the risk of heart disease, and you've just reduced your risk”, or “good news, you now may be 75% less likely to have a heart attack based on the improvement of your waist to hip ratio.” Other goals displayed on the lining of the fabric can relate to overall weight loss, and can be expressed by motivational messages such as, for example, “congratulations! You've lost another pound (or kilogram)”, or “maintaining a healthy weight can decrease risk of cancer of the breast, colon, uterus, and kidney-you're well on your way”, or, for example, “great job!, the risk of type 2 diabetes goes down as your weight goes down”.

For even greater health benefits, the use of supplemental additives or modifications of the non-refined food diet can be included along with abstinence from refined foods. The adjustment of the fat content of the meals within the invention can help stabilize certain conditions such as, for example, epilepsy and similar neuro-disorders. A rotation diet, where the total calories consumed in a week are varied on a set, repeating weekly schedule, can be implemented to reduce boredom from the restricted diet and prevent the person's metabolism from slowing. Rotation diets have been considered difficult to organize and comply with, but the use of the food kit in the invention allows for easier implementation of such a diet. The meals within the invention can be vegan, that is no animal products (including dairy), to help control pain by eliminating dietary triggers to pain found in animal products. A carbohydrate blocker can be added to the diet to further assist in reducing excess weight.

The invention uses particular apparatuses and methods to stabilize neurotransmitter levels, which are often initially decreased as the individual abstains from refined foods. The addiction to refined foods is often characterized by release of the neurotransmitter dopamine in response to eating refined food. The individual seeks the refined food to continue to stimulate neurotransmitter release. Many of the symptoms of withdrawal are caused by reduced neurotransmitter production due to the lack of the refined food stimulus, which the body has become dependent upon. The invention can use an apparatus for exercise in combination with the elimination of refined foods to stimulate pleasurable neurotransmitter release. The exercise equipment allows progressive recovery of functions compromised by the refined food addiction, such as, for example, strength, endurance, flexibility, breathing, and ability to stay engaged. As these functions return, the person is further motivated to maintain their diet regime and lifestyle changes. The invention can also include devices to measure the level of exercise such as a pedometer. The release of neurotransmitters stimulated by exercise also helps counter any decrease in neurotransmitter level as the body adjusts to the lack of refined food stimulus. As the drowsiness, fatigue, depression and lack of concentration that can accompany refined food addiction decreases, the ability to maintain an exercise program increases.

The invention can also use a stress monitor in combination with meditation to regulate neurotransmitter levels. As stress is reduced by meditation with the biofeedback from the monitor, pleasurable neurotransmitters, such as, for example, dopamine, are increased, helping to counter any initial decrease in neurotransmitter level as refined foods are eliminated from the diet.

Dietary supplements can be added to the non-refined food diet for their effect on mood and neurotransmitter functioning in people suffering from a refined food addiction. Since the refined food addict will be denied the use of refined food for their mood elevating effect, supplements can be helpful in stabilizing neurotransmitter level and elevating mood, thereby reducing the adverse symptoms of refined food withdrawal that might hinder abstinence from refined foods. One such supplement, for example, can be epigallow-catechin gallate, found in green tea extract, or HCA and 5-HTP, all of which may have mood elevating properties. Other supplements can stabilize blood glucose levels such as chromium and conjugated linoleic acid; reduce side effects during relapse such as carb-blockers; and reduce harm in those who cannot stop overeating such as starch-blockers derived from white kidney beans and hibiscus, as well as sugar blockers derived from L-arabinose and fat-blockers such as chitosan and other soluble fibers.

Since the food kit can be ordered and delivered to the person, it avoids the inconvenience and temptations associated with other diet programs where the individual has to go to a store to select diet items, often spending more time searching for appropriate foods and being tempted by less healthy foods. The invention can therefore include a method involving the production, preparation, picking, and packing of the foods kits. The preparation of the food kits can be accomplished in a system, for example, whereby food is delivered to the production facility, the food is cleaned, cut, steamed, and put into recyclable plastic packets (or possibly reusable metal containers or recyclable paper containers), the ingredients are held in bins in a frozen picking room, a computer-guided picker can pick food and seasoning items and drop it into the insulated shipping container in the order in which it is to be consumed, a calendar of use can be printed and dropped in the box, a checker can check the items, a sealing machine can seal the box and apply the shipping label, and the shipping container can be held in a freezer warehouse to be picked up at the end of the day by the overnight shipper. The food shipment can then be shipped by overnight services in reusable insulated containers with recyclable plastic utensils. The containers and utensils could then be shipped back to be recycled.

The method of providing the food kit is made convenient to facilitate the initiation of the non-refined food diet and encourage adherence to the diet. By avoiding the time in finding diet food and the temptation of being exposed to unhealthy food in grocery stores and restaurants, the method for providing the food kit increase adherence to the diet. The invention can include, for example, a process, whereby the person can order the kit, or reorder the kit when the food from the previous kit is almost exhausted, over the internet or by phone. The person can indicate food preferences. The person can then provides shipping instructions including address. The person can then fill out various health data questionnaires of interest in furthering research into refined food addiction. The person can then pay for the food and any associated equipment, and the shipment is then delivered. Related equipment that can be sent out with the shipment includes, but is not limited to, health monitoring equipment, whereby such health monitoring equipment can include, but is not limited to, a blood pressure cuff, a heart rate monitor, blood glucose measuring equipment, cholesterol measuring device, and a device that measures weight and percentage of body weight represented by fat. The shipment can further include a daily calendar that is used to plan a rotation diet. A means is also provided for the person to order the food kit from an internet website, which makes the ordering processes even more convenient and increases the likely compliance with the diet program.

The invention also includes a method for identifying a disorder related to the consumption of refined foods. Such identification is accomplished, for example, by having the person take an assessment questionnaire to determine their addictive behavior. Such a questionnaire uses questions from the previously discussed substance-related disorder questionnaires such as, for example, the DSM or ICD. In addition to taking responses to this questionnaire and scoring them based on whether the responses describe addictive behavior, this method examines the person's medical history, possibly looking at whether the person exhibited past medical problems with addictive substance like alcohol or tobacco to predict tendency to food addiction. This method can also test the person for disorder-related biomarkers, such as, for example, abnormal glucose levels, and can use neuron-imaging devices to view the size of the dopamine field within the brain to detect any abnormalities. Food addicts often have an increased portion of their brain that contains elevated levels of the neurotransmitter dopamine, as do individuals addicted to drugs and alcohol.

The invention uses the assessment questionnaire to allow the person to determine the addictive properties of individual food items, which would motivate the individual to be especially careful in avoiding particular foods. This educational and motivational effect is advanced by first allowing the person to see the initial effect of abstaining from refined foods by way of their responses to the questionnaire, which could then be scored to indicate the degree of addictive behavior, then assessing the effect of consuming a specific food item by completing and scoring the assessment questionnaire after such consumption, and then confirming this effect by again abstaining from refined food and again taking the assessment questionnaire and scoring the responses to indicate addictive behavior. The person can become educated as to the addictive properties of particular foods by seeing the results of the questionnaire at these time periods. With this information, the person determines the extent of their addiction to specific refined foods and experience the consequences of using refined foods. As the person experiences the impact of particular foods on their body they can develop aversion and attraction behaviors based on these associations.

The food kit can be used after the receipt of a shipment containing an insulated shipping container. The container can have a removable section for about 35 servings of protein, for example, a second removable section for about 21 servings of carbohydrate, for example, a third removable section for about 21 servings of vegetables, for example, and a fourth removable section for about 14 servings of fruit, for example. Alternatively, the section can contain for example, about 15 servings of protein, about 9 servings of carbohydrate, about 9 servings of vegetables, and about 6 servings of fruit. Alternatively, the section could contain, for example, about 70 servings of protein, about 42 servings of carbohydrate, about 42 servings of vegetables, and about 28 servings of fruit. Each section can be color coordinated with the color of the type of food. The container can have slots for pre-measured servings of fats, slots for optional seasonings, and a slot on the cover for a shipping label. The container can also have nylon handles on the side for convenient carrying.

The sections of the insulated shipping container can contain, for example, frozen food packets with about 35 servings of protein, about 21 servings of carbohydrate, about 21 servings of vegetables, and about 14 servings of fruit, enough food to last about one week. Alternatively, the servings can be, for example, sufficient to last from about 3 days to about one month or longer. The food can be minimally prepared with only the application of cutting and enough heat to make it edible. One embodiment, there is only one food class in each packet. Each serving can be individually encased, for example, in a sealed clear plastic packet or some other appropriate container. Each packet can be color coded for the type of food enclosed. Each packet can have the name of the food enclosed as well as the name of the appropriate optional seasoning. Each packet can be labeled with a bar code that can be scanned. Each packet can have attached an appropriate plastic utensil. Instructions can also be provided for combining the food packets into meals.

The shipment can contain, for example, a day pack with about 5 slots for about 5 meals, constructed from any appropriate material, with a zipped lid with both a short and long strap that can be carried by hand or over the shoulder. There can be, for example, about 2 slots for about 2 breakfast/snacks composed of one serving of protein and one serving of fruit. The slots can be marked with the color of the appropriate food so the breakfast/snack slots can be colored with a red stripe and a yellow stripe, for example. About 3 slots can be for about 3 lunch/dinners composed of one serving of protein, one serving of carbohydrate, and one serving of vegetable. These slots can be colored, for example, with a red stripe for the protein, a white stripe for the carbohydrate, and a blue stripe for the vegetable. Various embodiments of the invention can include, for example, the day pack, and the number of meals in any day pack could vary with more or fewer meals in the day pack depending on the anticipated daily needs of the individual.

The food kit can be used by a person in such a way that, upon receipt of the shipment, the person can unload the kit into the home freezer. At night, the person can remove from the home freezer a serving of protein and a serving of fruit for a breakfast/snack to be eaten the next morning. Each morning, the person can eat the thawed serving of protein and fruit for breakfast. Each morning, for example, by matching the color of the stripes in the daypack with the colors on the food packets, the person can unload the appropriate food packets from the home freezer into the daypack to make about three lunch/dinners and about one breakfast/snack. During the morning, the food thaws so the person can eat it at lunch and dinner. Each morning and each evening, the person can collect health data and transmit it to a researcher/manufacturer. The food can be consumed over, for example, about seven days or a sufficient time to recover from refined food addiction, whereby after this period of time, person achieves withdrawal from addictive refined foods. Of course, the amount of food and amount of time the person requires to withdraw from refined food addiction varies from about several days to about a month or longer, depending on the individual and the severity of the addiction.

The abstinence from refined foods that the invention enables can be used to treat other health conditions that are impacted by the ingestion of refined foods. For example, diabetes, epilepsy, pain and obesity are all benefited from the improved health status and reduction in excess weight that is facilitated by the elimination of refined foods. The non-refined food diet, for example, helps put diabetes into long-term remission by restoring insulin receptor functioning.

Numerous other diseases and conditions can be treated by the invention, including but not limited to the following: abnormal triglycerides; accident prone; acid stomach; aches; acne; addiction; carbohydrate addiction; caffeine addiction; susceptibility to drug and alcohol addiction; adenosine receptor dysfunction; excessive adipose tissue accumulation; adrenal exhaustion; adrenaline (epinephrine): blood clotting dysfunction; adrenaline/insulin imbalance; adult-onset diabetes; aggressive behavior; accelerated aging; agoraphobia; alcohol abuse; alcoholism; aldosterone dysfunction; food allergies; skin allergies; gastrointestinal allergies; air borne allergies; Alzheimer's; amputation; anemia; inappropriate anger; anorexia; antisocial behavior; antioxidant dysfunction; anxiety; appendicitis; appetite dysfunction; arrhythmia; constriction of arteries; arthritis; asthma; arteriosclerosis; attention deficit disorder; attention deficit hyperactivity disorder; autism; autoimmune diseases; auto-immune thyroiditis; back pain; bacterial infections; behavioral disorders; beta-endorphin dysfunction; binge-eating; birth defects; blindness; bloating; blood circulation disorder; blood clotting dysfunction; elevated blood pressure; blood sugar instability; blood vessel constriction; bone loss; brain fatigue; brain fog; breast cancer; breast soreness; bronchitis; bulimia; bursitis; calcium deficiency/depletion; calcium metabolism dysfunction; cancer; breast cancer; Candida; carbohydrate-induced high blood fat; cardiovascular disease; cellulose fiber; central nervous system suppression; chemical allergies; elevated cholesterol; chronic depression; chronic fatigue; cigarette smoking; claustrophobia; codependency; cognition impairment; coldness in the extremities; colitis; colorectal cancer; colorectal tumors; poor communication skills; compulsive behavior; compulsive eating; concentration lack of; confused thinking; congestion; congestive heart failure; constipation; coronary artery occlusion; coronary vasospasm; excessive cortisol; cramps; excessive cravings; criminal behavior; Crohn's disease; cystic breast disease; cystitis; poor decision-making; dehydration; dementia; depression; DHEA; diabetes; diarrhea; digestive disturbances; disorientation; dopamine dysfunction; drowsiness; drug abuse; drug addiction; ear infections; eating disorders; eczema; emotional instability; encephalitis; endogenous opioid over stimulation; endometrial cancer; endometrial tumors; endometriosis; endorphin rush; unstable energy levels; epilepsy; esophageal cancer; inappropriate euphoria; exhaustion; eczema; fatigue; irrational fear; excessive hunger; fertility; fibrocystic breast disease; fibromyalgia; cold fingers; fluid retention; food allergies; bone fractures; free radicals; gallstones; gastritis; gastroesophageal reflux disease; gastrointestinal bleeding; gastrointestinal distress; cramping; diarrhea; glucose intolerance; gout; growing pain; gum; bleeding; gum disease; hay fever; headaches; hearing loss; heart attacks; heart disease; heart rhythm disturbance; heartburn; hemophilia; hemorrhage; hemorrhoids; high blood pressure; HIV infection; hives; hormone imbalance; hostile behavior; hyperactivity; hypercholesterolemia; hyperglycemia; hyperinsulinemia; hyperkinesis; hyperlipidemia; hypertension; hypercholesterolemia; hypoglycemia; hypothalamic hormone imbalance; hypothyroidism; immune suppression; nutrient absorption impairment; poor impulse control; inability to lose weight; inappropriate hunger; inattentiveness; infectious diseases; infertility; inflammatory bowel disease; influenza; insomnia; insulin; insulin imbalance; insulin resistance; iron absorption dysfunction; iron-deficiency anemia; irrational thinking; irregular menstrual periods; irregular heartbeat; irrational behavior; irritability; irritable bowel syndrome; Ischemia; jaw tension; joint pain; kidney disease; kidney stones; leaky gut syndrome; learning disorders; leukemia; light headedness/dizziness; lipid metabolism disorder; loneliness; low estrogen production; low self-esteem; lung congestion; lupus; magnesium deficiency; malignant hypertension; malnutrition; manic depression; medication or drug interactions; melatonin dysfunction; memory problems; menopause; menstrual irregularity; diminished mental acuity; mental illness; metabolic dysfunction; impaired absorption of minerals; miscarriage; mood disorders; mood swings; lack of motivation; excessive mucus; multiple sclerosis; muscle aches; musculoskeletal pain; myocardial infarction; nasal congestion; nausea; neck pain; nephrosis (kidney disease); nervous system damage; nervousness; neuroreceptor dysfunction; neuroreceptor downregulation; numbness; obesity; obsessive thinking; obsessive-compulsive disorder; osteoarthritis; osteoporosis; feeling of being overwhelmed; chronic pain; palpitations; pancreatitis; pancreatic cancer; panic attacks; paroxysmal atrial tachycardia; pathological emotional attachments; pediatric hypertension; irrational pessimism; renal disease; phobias; plaque formation; Polycystic ovarian syndrome; post traumatic stress disorder; loss of potassium; premenstrual syndrome; prostate cancer; psychosis; psychosomatic illness; racing heart beat; rashes; reactive hypoglycemia; impaired reasoning; restless leg syndrome; rheumatoid arthritis; ringing in the ears; rosaia; satiety disorder; schizophrenia; low self-confidence; low self-esteem; separation anxiety; serotonin dysfunction; decreased libido in sex; impaired sexual satisfaction; shakiness; shortness of breath; sinusitis; skin rash; sleep apnea; sleep deprivation; sleepiness; sleeplessness; excessive sneezing; steroid hormone dysfunction; stomach ache; stress; stroke; swelling; teeth clenching or grinding; tension headaches; thyroid disorders; thyroid dysfunction; tinnitus; TMJD (temporomandibular joint dysfunction); tobacco addiction; tooth decay; trembling/tremors; ulcerative colitis; ulcers; vaginal yeast infections; vaginitis; violent behavior; viral infections, vomiting; weight gain; weight loss; yeast infections; and yeast overgrowth.

### EQUIVALENTS

Other embodiments will be evident to those of skill in the art. It should be understood that the foregoing detailed description is provided for clarity only and is merely exemplary. The spirit and scope of the present invention are not limited to the above examples, but are encompassed by the following claims. All references cited herein are incorporated by reference in their entireties.

What is claimed is:

1. A method for treating a person afflicted with a disorder related to the consumption of refined foods, comprising:
a) using a food kit comprising prepared food that does not contain refined food products, wherein the kit comprises sufficient food to supply the nutritional requirements of the person during a specified period of time; and
b) eliminating the consumption of refined food products during the period of time, thereby treating the person afflicted with the disorder related to the consumption of refined foods.

2. The method of claim 1, wherein the disorder is Refined Food Addiction.

3. The method of claim 1, wherein the refined food product is selected from the group consisting of: plant products that have been concentrated, plant products that have been made into a powder, plant products that have been made into a syrup, plant products that have been made into a crystal, and combinations thereof.

4. The method of claim 1, further comprising the elimination of food products and additives selected from the group consisting of: salt, caffeine, fats exposed to high temperatures, and processed dairy products.

5. The method of claim 1, wherein the food kit comprises an apparatus that organizes prepared unrefined foods into storage compartments that separate food items into individual meals.

6. The method of claim 5, wherein each item of a particular type of food is enclosed in an individual container.

7. The method of claim 5, wherein each container is color-coded for the particular type of food, wherein the kit comprises instructions for matching the color coded containers to create meals by combining particular types of food.

8. The method of claim 5, further comprising a machine-readable bar code on each container.

9. The method of claim 1, wherein the food kit further comprises optional seasonings.

10. The method of claim 1, wherein the food kit comprises specifically apportioned meals.

11. The method of claim 10, wherein each meal is apportioned according to a static ratio of proteins to fruit, vegetables and starches.

12. The method of claim 1, further comprising
c) assessing eating behaviors during the period of time using DSM, ICD, or any other substance use disorder criteria;
i) obtaining responses to an assessment questionnaire that ranks the severity of addictive behavior using DSM, ICD, or any other substance abuse criteria as applied to the consumption of edible substances at the beginning of the method, at a later time point, and after the consumption of any refined food, comprising:
ii) recording the responses to the questionnaire obtained in i); and
iii) comparing the responses given to questionnaire at the beginning of the method to those given at later time periods.

13. The method of claim 1, further comprising a means to reorder the food kit when food is almost exhausted.

14. The method of claim 13, further comprising a means to alter the order of the initial food kit package in accordance to the food preferences of the person.

15. The method of claim 1, further comprising one or more health monitoring devices.

16. The method of claim 1, further comprising the use of a wireless, hand-held electronic device.

17. The method of claim 16, wherein the hand-held electronic device has telecommunication and computational capabilities.

18. The method of claim 17, wherein the hand-held electronic device performs one or more of the functions selected from the group consisting of: collecting data on food consumption, automatically reordering food kit packages when a previous food kit is close to exhausted, showing financial savings from use of food kit, providing ratings of restaurants and grocery stores, providing information on recommended meals for a particular diet, providing motivational feedback on the positive consequences of abstaining from refined foods, providing motivational feedback on the negative consequences of relapse in consuming refined foods and providing information on food allergies.

19. The method of claim 1, further comprising the use of adjustable clothing to measure waist and hip, wherein the clothing comprises goals displayed in the lining of the fabric in place of numbers.

20. The method of claim 1, wherein the food kit comprises food with adjustable fat levels.

21. The method of claim 1, wherein the food kit comprises means suitable for a rotation or vegan diet.

22. The method of claim 1, further comprising a carbohydrate blocker.

23. The method of claim 1, further comprising an apparatus for exercise.

24. The method of claim 1, further comprising a stress monitor to be used in combination with meditation.

25. The method of claim 1, further comprising dietary supplements

26. A method for providing a food kit for treating an eating-related substance use disorder; comprising
a) using an apparatus that, in response to a customer order, organizes prepared, unrefined foods into a food kit, wherein the food kit comprises multiple compartments that separate prepared foods into separate meals, and wherein the food kit comprises enough meals for a specified period of time; and
b) a means for the customer to order the food kit; and
c) delivery of the food kit to the customer,
whereby appropriate use of the delivered food kit treats the eating-related substance use disorder.

27. The method of claim 26, wherein the food kit is delivered overnight.

28. The method of claim 26, further comprising a means for the customer to reorder the food kit when food is almost exhausted.

29. The method of claim 26, further comprising providing health monitoring equipment.

30. The method of claim 29, wherein the health monitoring equipment comprises a blood pressure cuff.

31. The method of claim 29, wherein the health monitoring equipment comprises a heart rate monitor.

32. The method of claim 29, wherein the health monitoring equipment comprises blood glucose measuring equipment.

33. The method of claim 29, wherein the health monitoring equipment comprises a cholesterol measuring device.

34. The method of claim 29, wherein the health monitoring equipment comprises a device to measure weight and percentage of body weight represented by fat.

35. The method of claim 26, further comprising instructions for eating foods from the kit according to a daily calendar.

36. The method of claim 26, wherein the means for the customer to order the food kit is provided on an internet website.

37. A method for identifying a disorder related to the consumption of refined foods comprising one or more methods selected from the group consisting of: obtaining responses to an assessment questionnaire, evaluating a medical history, detecting disorder-related biomarkers and neuroimaging, wherein the size of the dopamine field is examined to detect abnormalities.

38. A method for measuring the addictive characteristics of a food, comprising
a) measuring of degree of food addiction using an assessment questionnaire;
b) adhering to a diet that eliminates all refined food items for a specified period of time;
c) measuring symptoms of food addiction after the specified period of time on the restricted diet;
d) reintroducing a single refined food item after the specified period of time;
e) measuring symptoms of food addiction at a time point after single refined food is reintroduced;
f) re-eliminating all refined foods from diet;
g) re-measuring symptoms of food addiction at a time point after all refined foods have again been eliminated from the diet; and
h) comparing the measurements of symptoms of food addiction,
thereby determining the severity of the addictive property of the specific refined food tested.

39. An apparatus that organizes prepared unrefined foods into a food kit wherein storage compartments separate food items into individual meals sufficient to last during the period of time.

40. The apparatus of claim 39, wherein each item of a particular type of food is enclosed in an individual container.

41. The apparatus of claim 40, wherein each container is color-coded for the particular type of food with instructions for matching the color coded containers to create meals by combining particular types of food.

42. The apparatus of claim 39, further comprising of a machine-readable bar code on each container.

43. The apparatus of claim 39, wherein the food kit further comprises optional seasonings.

44. The apparatus of claim 39, wherein the food kit comprises specifically apportioned meals.

45. The method of claim 44, wherein each meal is apportioned according to a static ratio of proteins to fruits, vegetables and starches.

46. Adjustable clothing comprising goals displayed in the lining of the fabric in place of numbers to measure the waist and hip thereby providing motivation for weight loss and a means for monitoring weight loss.

47. A method for treating adult epilepsy, comprising:
a) using a food kit comprising prepared food that does not contain refined food products such that the kit comprises sufficient food to supply the nutritional requirements of the person during a specified period of time; and
b) eliminating the consumption of refined foods during the period of time, thereby treating adult epilepsy.

48. A method for treating pain, comprising:
a) using a food kit comprising prepared food that does not contain refined food products such that the kit comprises sufficient food to supply the nutritional requirements of the person during a specified period of time; and
b) eliminating the consumption of refined foods during the period of time, thereby treating pain.

49. A method for treating obesity, comprising:
a) using a food kit comprising prepared food that does not contain refined food products such that the kit comprises sufficient food to supply the nutritional requirements of the person during a specified period of time; and
b) eliminating the consumption of refined foods during the period of time, thereby treating obesity.

50. A method for treating diabetes, comprising:
a) using a food kit comprising prepared food that does not contain refined food products such that the kit comprises sufficient food to supply the nutritional requirements of the person during a specified period of time; and
b) eliminating the consumption of refined foods during the period of time, thereby treating diabetes.
